(19) United States
(12) Patent Application Publication
(10) Pub. No.: US 2004/0171880 A1
(43) Pub. Date: Sep. 2, 2004

(54) Process for the production of amides

(76) Inventors: Jack S. Warren, Blountville, TN (US); David R. Westphal, Chanhassen, MN (US); Steve J. Zoubek, Otsego, MN (US)

Correspondence Address: POPOVICH & WILES, P.A., Suite 1902, IDS Center, 80 South 8th Street, Minneapolis, MN, 55402, US

(21) Appl. No.: 09/982,105

(22) Filed: Oct. 17, 2001

Related U.S. Application Data
(60) Provisional application No. 60/244,693, filed on Oct. 31, 2000.

(51) Int. Cl. C07C 231/02

(52) U.S. Cl. 564/139

(57) ABSTRACT

A process for the production of an amide comprising: (a) reacting R₁—CX and oxygen to form R₁—COOH, wherein the reacting occurs in the liquid or vapor phase and in the presence of a first catalyst, wherein X is a group that leaves upon oxidation, and wherein R₁ is phenyl, which is unsubstituted or substituted by one or more identical or different radicals selected from (C₁-C₁₂)-alkyl, (C₁-C₁₂)-alkoxy, (C₁-C₁₂)-alkanoyloxy, (C₁-C₁₂)-alkanoyl, amino, hydroxyl, —CH₂—O—(C₁-C₁₂)-alkyl, —NH—(C₁-C₁₂)-alkyl, —NH—CO—(C₁-C₁₂)-alkyl, or —S—(C₁-C₁₂)-alkyl; (b) separating the R₁—COOH from the mixture formed in step (a), wherein the R₁—COOH is maintained in a liquid or vapor phase; and (c) reacting the R₁—COOH maintained in the liquid or vapor phase from step (b) with an amine to form an amide, wherein the reacting occurs in the vapor phase and in the presence of a second catalyst.

This application claims the benefit of provisional application Serial No. 60/244,693, filed Oct. 31, 2000, the contents of which are hereby incorporated herein by reference.

### FIELD OF THE INVENTION

This invention relates to the preparation of amides. More particularly, this invention relates to a process for the production of amides using tube reactors.

### BACKGROUND OF THE INVENTION

N,N-di(n-lower alkyl)-meta-toluamides are useful as chemical and pharmaceutical intermediates, pharmaceuticals, agricultural agents and insecticides. In particular, N,N-di(ethyl)-meta-toluamide is widely used as an active ingredient in insect repellent formulations. One method of preparing N,N-diethyl-m-toluamide is a two-step process in which meta-toluic acid and phosphorus oxychloride are reacted in the liquid phase with diethylamine. One disadvantage of this method is the cost of phosphorus oxychloride and alkali needed to neutralize residual amounts of this reactant after completion of the first step. Another disadvantage is the generation of gaseous hydrogen chloride as a reaction product in the second step of the preparation.

U.S. Pat. No. 2,932,665 (Wagner) and U.S. Pat. No. 3,198,831 (Stryk) disclose processes for the preparation of N,N-diethyl-toluamides involving the reaction of a toluic acid with diethylamine in the vapor phase in the presence of silica gel, alumina or boron phosphate catalyst. U.S. Pat. No. 2,932,665 (Wagner) also teaches that liquid phase reaction of meta-toluic acid with diethylamine in the presence or absence of a dehydration catalyst to produce N,N-di(ethyl)meta-toluamide is marked by long reaction times plus generation of objectionable amounts of odor and color bodies and undesired by-products, and that the catalyzed and uncatalyzed reaction yields are substantially the same.

U.S. Pat. No. 3,825,596 (Naito et al.) discloses a liquid phase process for producing N,N-di(lower alkyl)-m(and p)-toluamides in which meta- (or para)-tolunitrile, a di(lower alkyl)amine and water are reacted at a temperature of 100° C. to 400° C., preferably in the presence of a catalyst selected from inorganic acids, organic acids, organic acid metal salts, metal halides, and peroxides. The preferred organic acid metal salts and metal halides are the acetates and chlorides of copper, zinc, cadmium, mercury, nickel, cobalt and lead. Care must be taken to remove ammonia, a reaction product, from the reaction system in order to obtain a satisfactory reaction yield. U.S. Pat. No. 3,825,596 (Naito et al.) teaches that the process described therein is not a dehydration reaction.

U.S. Pat. No. 4,133,833 (Hull) describes a single-step process for the production of N,N-di(ethyl)-meta-toluamide in which meta-toluic acid is contacted with diethylamine in the liquid phase in the presence of a catalyst at temperatures from 150° C. to 300° C. The water produced in the reaction is removed from the liquid phase during the reaction. The product is recovered by distillation.

In current processes for the production of N,N-di(ethyl)-meta-toluamide, the starting material, meta-toluic acid, is a solid. This solid is packaged and conveyed to a reaction vessel where it is mixed with diethylamine. The handling of meta-toluic acid can be problematic and is expensive. A need in the art remains for an efficient reaction method that avoids some of the handling problems of previously known methods for the production of high purity N,N-di(ethyl)-meta-toluamide.

### SUMMARY OF THE INVENTION

The invention provides a process for the production of an amide comprising: (a) reacting R₁—CX and oxygen to form R₁—COOH, wherein the reaction occurs in the liquid or vapor phase and in the presence of a first catalyst, wherein X is a group that leaves upon oxidation, and wherein R₁ is phenyl, which is unsubstituted or substituted by one or more identical or different radicals selected from (C₁-C₁₂)-alkyl, (C₁-C₁₂)-alkoxy, (C₁-C₁₂)alkanoyloxy, (C₁-C₁₂)-alkanoyl, amino, hydroxyl, —CH₂—O—(C₁-C₁₂)-alkyl, —NH(C₁-C₁₂)-alkyl, —NH—CO—(C₁-C₁₂)-alkyl, or —S—(C₁-C₁₂)-alkyl; (b) separating the R₁—COOH from the mixture formed in step (a), wherein the R₁—COOH is maintained in a liquid or vapor phase; and (c) reacting the R₁—COOH maintained in the liquid or vapor phase from step (b) with an amine to form an amide, wherein the reaction occurs in the vapor phase and in the presence of a second catalyst.

In a preferred embodiment, the invention provides a process for the production of N,N-di(ethyl)-meta-toluamide comprising: (a) reacting meta-xylene and oxygen to form meta-toluic acid, wherein the reaction occurs in the liquid or vapor phase and in the presence of a first catalyst; (b) separating the meta-toluic acid from the mixture formed in step (a), wherein the meta-toluic acid is maintained in a liquid or vapor phase; and (c) reacting the meta-toluic acid maintained in the liquid or vapor phase from step (b) with diethylamine to form N,N-di(ethyl)-meta-toluamide, wherein the reaction occurs in the vapor phase and in the presence of a second catalyst.

Additional features and advantages of the invention are set forth in the description which follows and in part will be apparent from the description. The objectives and other advantages of the invention will be realized and attained by the process for the production of an amide as particularly pointed out in the written description and claims.

It is to be understood that both the foregoing general description and the following detailed description are exemplary and explanatory and are intended to provide further explanation of the invention as claimed.

### BRIEF DESCRIPTION OF THE DRAWINGS

FIG. 1 is a schematic illustration of the apparatus of the present invention.

### DETAILED DESCRIPTION OF THE INVENTION

The invention provides a process for the production of an amide comprising: (a) reacting R₁—CX and oxygen to form R₁—COOH, wherein the reaction occurs in the liquid or vapor phase and in the presence of a first catalyst, wherein X is a group that leaves upon oxidation, and wherein R₁ is phenyl, which is unsubstituted or substituted by one or more identical or different radicals selected from (C₁-C₁₂)-alkyl, (C₁-C₁₂)-alkoxy, (C₁-C₁₂)alkanoyloxy, (C₁-C₁₂)-alkanoyl, amino, hydroxyl, —CH₂—O—(C₁-C₁₂)-alkyl, —NH(C₁-C₁₂)-alkyl, —NH—CO—(C₁-C₁₂)-alkyl, or —S—(C₁-C₁₂)-alkyl; (b) separating the R₁—COOH from the mixture formed in step (a), wherein the R₁—COOH is maintained in a liquid or vapor phase; and (c) reacting the R₁—COOH maintained in the liquid or vapor phase from step (b) with an amine to form an amide, wherein the reaction occurs in the vapor phase and in the presence of a second catalyst.

In a preferred embodiment, X is —H₃ (i.e., the compound is toluene). In another preferred embodiment R₁ is meta-methylphenyl. The amine is preferably a secondary amine, more preferably, diethylamine. Another preferred secondary amine is ethylhexylamine.

In a preferred embodiment, at least one of the first and second catalysts is a liquid catalyst. In another preferred embodiment, at least one of the first and second catalysts is a solid catalyst. Solid catalysts may be supported or unsupported and include one or more of MgO, TiO₂, ZrO₂, ZnO, CeO₂, Ce₂O₃, tungsten or molybdenum heteropolyacids, and hydroxyapatite. Liquid catalysts include cobalt octoate and copper octoate.

In a preferred embodiment, in step (c) the reaction of the R₁—COOH and the amine occurs in the presence of an added amount of the amide. In another preferred embodiment, the R₁—COOH in step (b) is maintained in a liquid phase and an amount of the amide is added to solubilize the R₁—COOH prior to the reaction in step (c). In a preferred embodiment, the weight ratio of the R₁—COOH and the amine is from about 1:1 to 4:1. In another preferred embodiment, the weight ratio of the R₁—COOH and the amine is about 2:1. In a preferred embodiment, the weight ratio of the R₁—COOH, the amine, and the added amount of amide is about 2:1:2.

In a preferred embodiment, at least one of the reactions in steps (a) and (c) takes place in a tube reactor; in a more preferred embodiment, the reactions in steps (a) and (c) takes place in tube reactors. In a preferred embodiment, at least one of the reactions in steps (a) and (c) takes place in two or more tube reactors connected in parallel. In a preferred embodiment, the process is a continuous process.

The invention provides a process for the production of N,N-di(ethyl)meta-toluamide comprising: (a) reacting meta-xylene and oxygen to form meta-toluic acid, wherein the reaction occurs in the liquid or vapor phase and in the presence of a first catalyst; (b) separating the meta-toluic acid from the mixture formed in step (a), wherein the meta-toluic acid is maintained in a liquid or vapor phase; and (c) reacting the meta-toluic acid maintained in the liquid or vapor phase from step (b) with diethylamine to form N,N-di(ethyl)meta-toluamide, wherein the reaction occurs in the vapor phase and in the presence of a second catalyst.

The preferred catalysts are those described above and below.

In a preferred embodiment, in step (c) the reaction of the meta-toluic acid and the diethylamine occurs in the presence of N,N-di(ethyl)-meta-toluamide.

In another preferred embodiment, the meta-toluic acid in step (b) is maintained in a liquid phase and an amount of the N,N-di(ethyl)-meta-toluamide is added to solubilize the meta-toluic acid prior to the reaction in step (c). In another preferred embodiment, the weight ratio of the meta-toluic acid and the diethylamine is from about 1:1 to 4:1. In another preferred embodiment, the weight ratio of the meta-toluic acid and the diethylamine is about 2:1. In yet another preferred embodiment, the weight ratio of the meta-toluic acid, the diethylamine, and the added amount of N,N-di(ethyl)-meta-toluamide is about 2:1:2.

In a preferred embodiment, at least one of the reactions in steps (a) and (c) takes place in a tube reactor; in a more preferred embodiment, the reactions in steps (a) and (c) takes place in tube reactors. In a preferred embodiment, at least one of the reactions in steps (a) and (c) takes place in two or more tube reactors connected in parallel. In a preferred embodiment, the process is a continuous process.

In another preferred embodiment, the invention provides a continuous tube reactor process for the production of amides by the partial oxidation of an alkyl benzene to a carboxylic acid immediately followed by reaction with an amine to form to an amide. Continuous liquid or vapor phase processing results in the efficient production of variously substituted amides and imides. In a preferred embodiment, meta-xylene is oxidized in the presence of a catalyst to meta-toluic acid, which flows in a continuous process to mix and react with a desired amine at elevated temperature and pressure in the presence of a catalyst. Reaction with diethylamine forms N,N-di(ethyl)-meta-toluamide. In addition, N,N-di(ethyl)-meta-toluamide is present as a solvent for the meta-toluic acid.

The invention provides a method for the continuous vapor phase processing of reactants results in efficient production of variously substituted amides and imides. One amide of particular commercial interest is N,N-di(ethyl)-meta-toluamide (also known as “DEET”), due to its use in insect repellent formulations.

In an embodiment of this invention, a carboxylic acid is formed by the catalyzed oxidation of a substituted benzene in accordance with the following reaction:

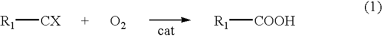

R₁—CX may be variously substituted alkyl benzenes, such as ortho-, meta-, or para-toluene. Therefore R₁ is, for example, phenyl or alkyl phenyl. There may be one or more alkyl groups, which are linear or branched alkyls comprising C₂ to C₁₂. The phenyl substituents may also include an organic group containing a hetero atom (e.g., N, S, O), provided the hetero atom does not interfere with the reaction with the amine. In a preferred embodiment, R₁ is methyl benzene (i.e., toluene) and is ortho-, meta-, or para- to the carboxylic acid position. In a more preferred embodiment, the methyl group is meta to the carboxylic acid position.

The catalyst to produce the acid may be a homogeneous or a heterogeneous catalyst. That is, the catalyst may be a compound in a liquid form that mixes with the hydrocarbon or it may be a solid phase catalyst as described further below.

In the process of this invention, a carboxylic acid and an amine form an amide reaction product. In the continuous process of this invention, the carboxylic acid is fed to a tank where it is solubilized by the amide.

Though other solvents could be used to solubilize the carboxylic acid, use of the reaction product to solubilize the acid eliminates the production of undesirable by-products and minimizes additional steps for the removal of solvents. The solubilized carboxylic acid and an amine are heated to form the gaseous phase and fed into a catalytic tube reactor where they react to produce the product according to the following reaction:

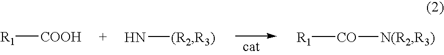

Preferably, the catalyst is a solid phase, as described further below.

Alternatively, a dicarboxylic acid may be reacted with NH(R₂,R₃) or NH₂R₂ to produce the imide, according to the following reaction:

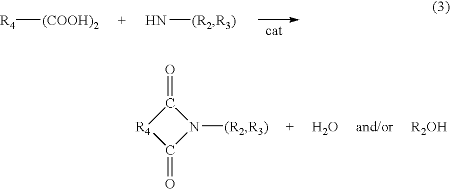

R₄ may be C₂-C₆ alkyl, C₂-C₆ alkenyl, or phenyl. Ortho-dicarboxylic acid benzene can be reacted to produce phthalimide.

Suitable amines include primary, secondary and tertiary amines that will react with a carboxylic acid to form an amide. This includes amines having a boiling point below about 200° C., preferably below about 160° C. Preferred amines of the formula HN(R₂,R₃) are those wherein R₂ is selected from one of hydrogen, C₁ to C₂₁ alkyl or alkenyl, aryl, and substituted aryl, and R₃ is selected from one of C₁ to C₂₁ alkyl or alkenyl, aryl, and substituted aryl. Preferred amines include diethyl amine (i.e., R₂ and R₃ are both ethyl) and ethylhexylamine.

In a preferred embodiment for the production of DEET, the reactions are:

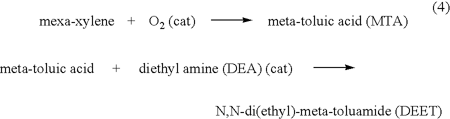

The reactions occur in the presence of a catalyst.

Preferably, the carboxylic acid reactant is prepared in-line and fed into a tank. In a preferred embodiment for the continuous production of N,N-di(ethyl)-meta-toluamide (DEET), meta-toluic acid (MTA) is first produced by the oxidation of meta-xylene in the presence of a catalyst. The production of meta-toluic acid in line provides for a more cost efficient production of the amide.

In the following preferred embodiment, the carboxylic acid and the amine are held in separate feed tanks, and the meta-toluic acid is solubilized by N,N-di(ethyl)-meta-toluamide in a weight ratio of about 1:1. The liquid reactants flow through lines into heaters and preferably are separately preheated to the gaseous phase. They are then combined in the gaseous phase and introduced into a tube reactor containing catalyst. Alternatively, the reactants may be introduced into the tube reactor as liquids and heated to vaporize there.

The reactants are fed into the catalytic reactor at elevated temperatures (ranging from about 100 to 400° C.) and elevated pressure, (ranging from 50 to 500 psi (344.7 to 3447 MPa)). The gas phase reactants mix together in weight ratio of about 1:4 (i.e., the ratio of diethyl amine to meta-toluic acid blend) and flow into the reactor. This ratio is believed to be an optimum ratio for this particular reaction, and is determined empirically. The optimum ratio will vary depending upon the particular carboxylic acid and amine used.

The reactants are typically heated by heaters as they flow to the reactor, but the tanks may also be heated. Preheating can reduce any variation in temperature within the reactor, thus providing for a more uniform reaction environment.

Tube reactors useful in the practice of this invention are preferably conventional stainless steel catalytic tube reactors that are filled with various combinations of inert filler material and a catalyst. Inert filler material may comprise glass beads between about 3 to 10 millimeters (mm) in diameter, or may include one or more of stainless steel beads, lava rock and sand. If the catalyst is a homogeneous catalyst, as in, for example, the oxidation of meta-xylene to meta-toluic acid, the tube reactor is filled only with an inert material, preferably glass beads. The filler material prevents laminar flow and promotes both turbulent flow and uniform exposure to the catalyst.

If the catalyst is a heterogeneous catalyst, as in the reaction between meta-toluic acid and diethylamine, the tube reactor may contain both the catalyst and inert filler material. At least a portion of the reactor is filled with a catalytic material. Various catalysts known in the art are useful in the production of amides and imides, and typically include a support that has been impregnated with a catalyst. Preferred catalysts and supports are described further below.

The reactants are fed through lines equipped with feed pumps into heaters and into the catalytic tube reactor. The reactants exit their tanks as liquids, or in the case of the meta-toluic acid, it exits its tank in a liquid solution of N,N-di(ethyl)-meta-toluamide. The liquid solution passes through a heater, which increases the temperature of the reactants to an optimum reaction temperature. Any heater effective at heating vapor phase components to temperatures up to about 400° C. is useful in the practice of this invention, but preferably the heater is an electric heater such as a superheater or vaporizer, available from AccuTherm, Inc. of Monroe City, Mo.

The heated reactants flow into the tube reactor where they are exposed to the catalyst and react to produce the desired products. Preferably, reactants are fed into the bottom of the reactor so that the reactant materials flow upwardly through the reactor. To minimize undesired co-products as well as “coking” of the catalyst and thus “plugging” of the reactor, the optimum reaction temperature of the reactant feed stream is determined. It may also be desirable to heat the catalyst bed to such optimum temperature prior to the introduction of the reactant materials.

In addition, the apparatus is equipped with a means to regenerate the catalyst in the tube reactor. When regeneration is desired, air/O₂/N₂ or other gases known to those skilled in the art is caused to flow through the reactor heated to about 550° C. Typically catalyst regeneration is carried out in about 8 to 12 hours.

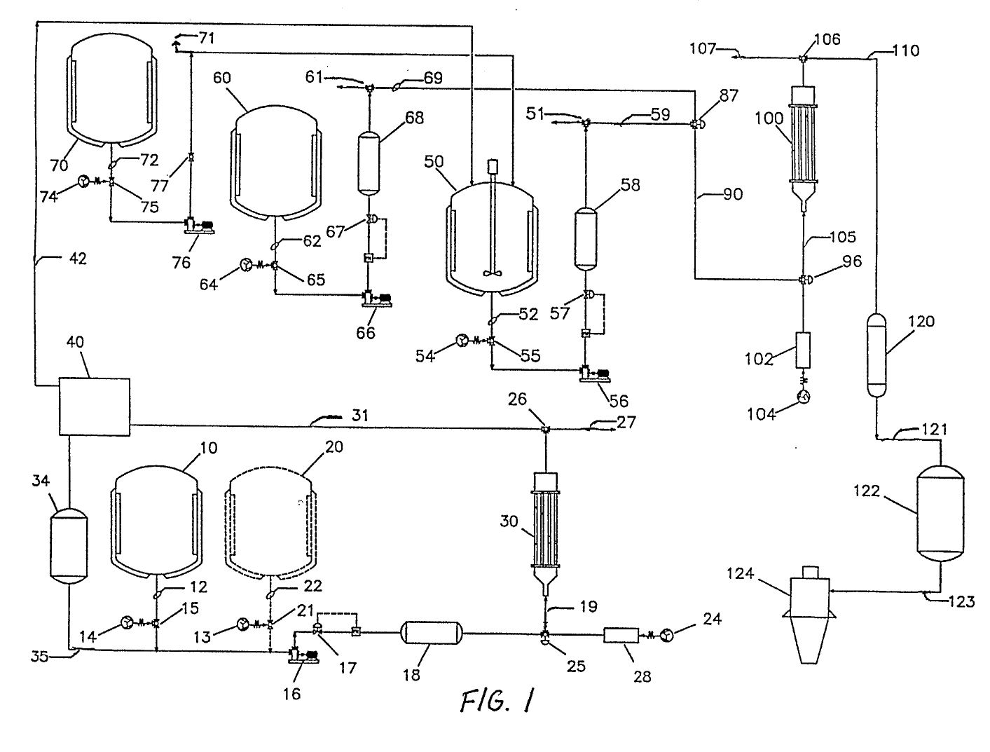

Further, it may be desirable to regenerate the catalyst bed in a tube reactor while the reaction is taking place. In this case, at least two tube reactors are connected in parallel, so that the reactants can flow through one tube reactor while another reactor undergoes regeneration. For the sake of simplicity, only one tube reactor, as described below, is shown in FIG. 1.

Turning now to FIG. 1, the method of this invention is carried out in apparatus 1. Meta-xylene is held in feed tank 10 and supplied with outlet conduit 12, which is provided with optional nitrogen purge source 14 and valve 15. Meta-xylene flows through conduit 12 to feed pump 16, which pumps the meta-xylene into heater 18, and heats the meta-xylene to about 250° C. Between the feed pump and heater 18 is valve 17, which controls the flow rate of the meta-xylene into the heater.

Optional catalyst feed tank 20 contains liquid catalyst. Feed tank 20 is supplied with outlet conduit 22 and valve 21. Conduit 22 joins conduit 12 leading from tank 10 and into feed pump 16. When liquid catalyst is used in the reaction, it is added in small quantities (i.e., parts-per-million) immediately before it enters the tube reactor. Typically no heater is necessary for the liquid catalyst. Meta-xylene flows from its tank into heater 18 and then into reactor 30. When a liquid catalyst is used, reactor 30 is filled with inert filler to promote uniform heating as well as turbulent flow of reactants. When liquid catalyst is not used, solid catalyst and inert filler fill reactor 30.

Meta-xylene exits heater 18 via conduit 12 which joins conduit 19 for introduction of the meta-xylene and liquid catalyst into tube reactor 20. Oxygen for the reaction is provided by gas source 24. O₂-enriched air flows through heater 28 to be heated to a desired temperature (150° C. to 350° C.) before flowing into conduit 19 and tube reactor 30. O₂-enriched air (air/O₂) flows into reactor 30 via conduit 19 by opening valve 25. Gas source 24 provides air/O₂/N₂, which is used to regenerate solid catalyst or to purge a line as desired.

During the oxidation of meta-xylene to meta-toluic acid, formation of the aldehyde occurs. To get a complete reaction, the aldehyde should be removed from the acid. Also, it is preferred that the aldehyde be recycled. The meta-toluic acid exits the reactor via conduit 31 provided with valve 26 and vent 27. Meta-toluic acid and any undesired aldehyde flows via conduit 31 into reboiler 40 where it is heated and separated. The aldehyde flows into recycle tank 34 and the meta-toluic acid flows into tank 50. The aldehyde then flows via conduit 35 from tank 34 and recombines with the meta-xylene feed stream in conduit 12.

Feed tank 70 contains recycled amides and imides. Though not shown, tank 70 is provided with a line leading from a distiller/separator to provide additional N,N-di(ethyl)-meta-toluamide as the reaction proceeds. N,N-di(ethyl)-meta-toluamide flows from tank 70 via conduit 72 which is fitted with nitrogen purge valve 75 leading to source of nitrogen gas 74. The N,N-di(ethyl)-meta-toluamide flows into feed pump 76 which pumps it via conduit 72 to the meta-toluic acid feed tank 50 to provide a solvent for the meta-toluic acid. Conduit 72 leading from feed pump 76 is also equipped with valve 77 so that flow can be shut off when desired. It also may be desirable to purge conduit 72 and pump 76 via vent 71 when needed or desired with the nitrogen gas.

Feed tank 60 contains diethylamine in a liquid state. The diethylamine flows out via conduit 62 which is fitted with nitrogen purge valve 65 leading to source of nitrogen gas 64. The diethylamine feeds into feed pump 66 and then to heater 68, where it is heated to between 100 and 400° C. The reactant remains within heater 68 for a time sufficient to convert it to its gaseous state. Between pump 66 and heater 68 is valve 67 which controls flow into the heater. It may be desirable to purge conduit 62, pump 66, and heater 68 via vent 61 when needed or desired with the nitrogen gas.

Molten meta-toluic acid is added into the N,N-di(ethyl)-meta-toluamide in feed tank 50 at inlet 49 via conduit 42, in a manner known to those skilled in the art to minimize or eliminate crystallization or solidification of the acid. Feed tank 50 is prepared to receive the meta-toluic acid by first adding to it N,N-di(ethyl)-meta-toluamide; thus the meta-toluic acid flows through the amide and mixes and solubilizes completely. The meta-toluic acid flows into the tank at an elevated temperature, at least about 112° C., in order to remain liquid. Typically tank 50 can be heated. Meta-toluic acid flows out of feed tank 50 via conduit 52 which is fitted with nitrogen purge valve 55 leading to source of nitrogen gas 54. The meta-toluic acid feeds into feed pump 56 and then to heater 58, where it is heated to between 100 and 400° C. It remains in heater 58 for a time sufficient to convert to its gaseous state. Between pump 56 and heater 58 is valve 57 which controls flow to the heater. It may be desirable to purge conduit 52, pump 56, and heater 58 when needed or desired with the nitrogen gas. The purge gas flows out via vent 51.

The reactants exit heaters 58 and 68 via lines 59 and 69, respectively. The reactants are now in a gaseous state and they combine at line 90, fitted with valve 87. Line 90 joins conduit 105 feeding into tube reactor 100. The pressure of the reactant stream as it enters the tube reactor is greater than ambient, and typically ranges from about 50 to about 500 psi ((344.7 to 3447 MPa)).

Tube reactor 100 is loaded with a suitable catalyst. For example, one suitable catalyst is titania on an alumina support. A surface area (for the alumina support) of 180 to 200 m²/g with a catalyst loading of about 20 weight percent results in conversions of greater than 80%, reaction selectivity of greater than 90% with reaction times in the range of about 4 hours. Another suitable catalyst is a tungsten heteropolyacid (H₃PW₁₂O₄₀) on silica. Using a 40 wt % tungsten heteropolyacid on silica results in reaction selectivity of 90 to 95% and conversion of 65 to 70% at 300 to 320° C. with reaction times of about 10 minutes.

Suitable catalysts for the reaction of diethylamine and methyltoluic acid to form N,N-di(ethyl)-meta-toluamide include magnesium oxide (MgO) and/or titania (TiO2) on alumina supports, tungsten heteropolyacids on silica supports and/or hydroxyapatites. Catalysts are described in detail below.

Weight hourly space velocity (WHSV) is the mass of product per hour divided by the mass of catalyst. In the apparatus shown for a tube reactor of ranging in diameter from 1 to 20 inches (2.5 to 50.8 cm), the WHSV ranges from 1 to 20.

Conduit 105 is also provided with source of air/O₂/N₂ 104 and heater 102. The air/O₂/N₂ is used when it is desired to regenerate the catalyst bed in the tube reactor. That is, reactant flow is terminated by closing valve 87 and valve 96 is opened to permit flow of regeneration gases. Reactor 100 is heated to about 550° C. with the desired gas or gases flowing through it to regenerate the catalyst. A regeneration cycle may require 8 to 12 hours to complete, and depends upon the volume of catalyst bed and degree of coking or contamination of the catalyst.

The amide exits tube reactor 100 via conduit 110 provided with valve 106 and vent 107. Product flows through conduit 110 into heat exchanger 120, which condenses it. The product flows via conduit 121 into receiving tank 122 and exits receiver tank 122 via conduit 123 into distiller/separator 124 where the product is purified. Distiller/separator 124 may be any separating apparatus that permits separation of the desired product from reaction by-products or residue, such as a fractionating column. The distiller/separator 124 typically is provided with a return line (not shown) to feed tank 70 to provide additional N,N-di(ethyl)-meta-toluamide as solvent for the meta-toluic acid during the production run.

Catalysts

For the production of a carboxylic acid from the above-described method, any suitable homogeneous catalyst is preferred. Such catalysts include metal compounds that are soluble in the starting material and they may be used at concentrations ranging from 200 to 2000 ppm. Suitable catalysts include cobalt and copper esters, such as cobalt and copper octoate. Other suitable homogeneous catalysts include soluble metal compounds selected from groups IB to VIIIB of the periodic table, i.e., salts of the transition metals, including such compounds as nitrates, perchlorates, halides, and sulfates. Soluble metallocenes may also be suitable.

For the production of the amide, solid catalysts preferably are used. Solid catalysts are typically those in which a catalyst is applied to a high surface area support material. Alternatively, the high surface area support material may be the catalyst. In general, metals or metal oxides selected from groups IB to VIIIB of the periodic table may be useful in the practice of this invention. Suitable catalysts include, but are not limited to, metal or metal oxides such as the oxides of magnesium (MgO), titanium (TiO₂), zirconium (ZrO₂), zinc (ZnO), cerium (CeO₂ or Ce₂O₃), or other lanthanides, and acid catalysts such as molybdenum or tungsten heteropolyacids and phosphate compounds such as the hydroxyapatites.

Tungsten heteropolyacid (H₃PW₁₂O₄₀) on silica was prepared by dissolving the corresponding hexahydrate in water to form a 0.5 gram/ml solution. This solution was used to impregnate a silica support material. The catalyst was dried at 110° C. for about 12 hours, then calcined in air at 300° C. for about 3 hours.

Hydroxyapatites include, for example, calcium phosphates of general formula Ca₍₁₀₋ₓ₎Hₓ(PO₄)₆(OH)₍₂₋ₓ₎, where x ranges from 0 to 2. A calcium phosphate wherein x=0 was prepared by the method described by Bett et al. in J. Amer. Chem. Soc., 89, 5535-5541 (1967). This method involves the addition of concentrated orthophosphoric acid to a saturated solution of Ca(OH)₂. Using a reactant ratio of ˜1/1/0.4 (DEA/MTA/DEET), and a reaction temperature of about 300° C., the reaction carried out with this calcium phosphate catalyst resulted in greater than about 60% conversion and reaction selectivity of about 90%.

Various catalyst supports are available from manufacturers such as United Catalyst Industries having various chemical composition, porosity, density, effective surface area, shape, size and cross section. Suitable catalyst supports include alumina, silica, zirconia and mixtures thereof as well as naturally occurring clays such as montmorillonite or kaolin. These support materials generally have effective surface areas ranging from about 20 to 500 m² per gram. Suitable substrates for use in the present invention include substrates such as titania (TiO₂), aluminum oxide (Al₂O₃), zinc oxide (ZnO), zirconium oxide (ZrO₂), silica (SiO₂), or combinations of these oxides.

In a preferred embodiment, a solution of the catalyst or a precursor to the catalyst is applied to an effective surface area of a support material such that there is substantially a theoretical monolayer of desired catalyst present. Further heat treatment of a catalyst precursor forms the catalyst. It has been found that a theoretical monolayer of catalyst optimizes the catalysis conditions. “Theoretical monolayer” refers to a film or layer of a material (catalyst) on a surface at a thickness of one molecule. As used herein, a substantially theoretical monolayer shall mean plus or minus 10% of a theoretical monolayer.

When applied, the catalyst or catalyst structure is expressed as weight percent of catalyst per unit weight of catalyst support. Conventionally, standard weight percentages of catalyst per unit weight of the support are prepared. For example, a 5% catalyst comprises 0.05 grams of catalyst per gram of catalyst support. Catalysts of 5%, 10% and other multiples of 5% are commonly prepared. This is accomplished by techniques including the incipient wetness method and spray or tumble-drying, as known to those of skill in the art.

Although conventional catalyst supports have effective surface areas ranging from about 20 to 500 m² per gram of support, the preferred catalyst support material for use in the production of amides and imides in the present invention has an effective surface area of about 20 to 500 m² per gram of support, more preferably about 100 to 300 m² per gram and most preferably about 180 to 200 m² per gram of support.

The above description and accompanying drawing are provided for the purpose of describing embodiments of the invention and are not intended to limit the scope of the invention in any way. It will be apparent to those skilled in the art that various modifications and variations can be made in the process for the production of an amide without departing from the spirit or scope of the invention. Thus, it is intended that the present invention include the modifications and variations of this invention provided they come within the scope of the appended claims and their equivalents.

What is claimed is:

1. A process for the production of an amide comprising:
(a) reacting R₁—CX and oxygen to form R₁—COOH, wherein the reacting occurs in the liquid or vapor phase and in the presence of a first catalyst, wherein X is a group that leaves upon oxidation, and wherein R₁ is phenyl, which is unsubstituted or substituted by one or more identical or different radicals selected from (C₁-C₁₂)-alkyl, (C₁-C₁₂)-alkoxy, (C₁-C₁₂)alkanoyloxy, (C₁-C₁₂)-alkanoyl, amino, hydroxyl, —CH₂—O—(C₁-C₁₂)-alkyl, —NH—(C₁-C₁₂)-alkyl, —NH—CO—(C₁-C₁₂)-alkyl, or —S—(C₁-C₁₂)-alkyl,
(b) separating the R₁—COOH from the mixture formed in step (a), wherein the R₁—COOH is maintained in a liquid or vapor phase, and
(c) reacting the R₁—COOH maintained in the liquid or vapor phase from step (b) with an amine to form an amide, wherein the reacting occurs in the vapor phase and in the presence of a second catalyst.

2. The method of claim 1, wherein X is —H₃.

3. The method of claim 1, wherein R₁ is meta-methylphenyl.

4. The method of claim 1, wherein the amine is a secondary amine.

5. The method of claim 1, wherein the amine is diethylamine.

6. The method of claim 1, wherein the amine is ethylhexylamine.

7. The method of claim 1, wherein at least one of the first and second catalysts is a liquid catalyst.

8. The method of claim 1, wherein at least one of the first and second catalysts is a solid catalyst.

9. The method of claim 1, wherein at least one of the first and second catalysts is selected from one or more of MgO, TiO₂, ZrO₂, ZnO, CeO₂, Ce₂O₃, tungsten heteropolyacid, hydroxyapatite, cobalt octoate, and copper octoate.

10. The method of claim 1, wherein at least one of the first and second catalysts is TiO₂.

11. The method of claim 1, wherein at least one of the first and second catalysts is cobalt octoate.

12. The method of claim 1, wherein at least one of the first and second catalysts is tungsten heteropolyacid.

13. The method of claim 1, wherein at least one of the first and second catalysts is hydroxyapatite.

14. The method of claim 1, wherein in step (c) the reaction of the R₁—COOH and the amine occurs in the presence of an added amount of the amide.

15. The method of claim 1, wherein the R₁—COOH in step (b) is maintained in a liquid phase and an amount of the amide is added to solubilize the R₁—COOH prior to the reacting in step (c).

16. The method of claim 1, wherein the weight ratio of the R₁—COOH and the amine is from about 1:1 to 4:1.

17. The method of claim 1, wherein the weight ratio of the R₁—COOH and the amine is about 2:1.

18. The method of claim 15, wherein the weight ratio of the R₁—COOH, the amine, and the added amount of amide is about 2:1:2.

19. The method of claim 1, wherein at least one of the reacting in steps (a) and (c) takes place in a tube reactor.

20. The method of claim 1, wherein the reacting in steps (a) and (c) takes place in tube reactors.

21. The method of claim 1, wherein at least one of the reacting in steps (a) and (c) takes place in two or more tube reactors connected in parallel.

22. The method of claim 1, wherein the process is a continuous process.

23. A process for the production of N,N-di(ethyl)-meta-toluamide comprising:
(a) reacting meta-xylene and oxygen to form meta-toluic acid, wherein the reacting occurs in the liquid or vapor phase and in the presence of a first catalyst,
(b) separating the meta-toluic acid from the mixture formed in step
(a), wherein the meta-toluic acid is maintained in a liquid or vapor phase, and
(c) reacting the meta-toluic acid maintained in the liquid or vapor phase from step (b) with diethylamine to form N,N-di(ethyl)-meta-toluamide, wherein the reacting occurs in the vapor phase and in the presence of a second catalyst.

24. The method of claim 23, wherein at least one of the first and second catalysts is a liquid catalyst.

25. The method of claim 23, wherein at least one of the first and second catalysts is a solid catalyst.

26. The method of claim 23, wherein at least one of the first and second catalysts is selected from one or more of MgO, TiO₂, ZrO₂, ZnO, CeO₂, Ce₂O₃, tungsten heteropolyacid, hydroxyapatite, cobalt octoate, and copper octoate.

27. The method of claim 23, wherein at least one of the first and second catalysts is TiO₂.

28. The method of claim 23, wherein at least one of the first and second catalysts is cobalt octoate.

29. The method of claim 23, wherein at least one of the first and second catalysts is tungsten heteropolyacid.

30. The method of claim 23, wherein at least one of the first and second catalysts is hydroxyapatite.

31. The method of claim 23, wherein in step (c) the reacting of the meta-toluic acid and the diethylamine occurs in the presence of an added amount of N,N-di(ethyl)-meta-toluamide.

32. The method of claim 23, wherein the meta-toluic acid in step (b) is maintained in a liquid phase and an amount of the N,N-di(ethyl)-meta-toluamide is added to solubilize the meta-toluic acid prior to the reacting in step (c).

33. The method of claim 23, wherein the weight ratio of the meta-toluic acid and the diethylamine is from about 1:1 to 4:1.

34. The method of claim 23, wherein the weight ratio of the meta-toluic acid and the diethylamine is about 2:1.

35. The method of claim 32, wherein the weight ratio of the meta-toluic acid, the diethylamine, and the added amount of N,N-di(ethyl)-meta-toluamide is about 2:1:2.

36. The method of claim 23, wherein at least one of the reacting in steps (a) and (c) takes place in a tube reactor.

37. The method of claim 23, wherein the reacting in steps (a) and (c) takes place in tube reactors.

38. The method of claim 23, wherein at least one of the reacting in steps (a) and (c) takes place in two or more tube reactors connected in parallel.

39. The method of claim 23, wherein the process is a continuous process.
